SEQUENCE LISTING (14 sequences)

SEQ ID NO: 1 - DNA, 18 bp, Artificial Sequence
  Description of Artificial Sequence Synthetic primer
cgctttcccaacgtcatt

SEQ ID NO: 2 - DNA, 20 bp, Artificial Sequence
  Description of Artificial Sequence Synthetic primer
agagtttgatymtggctcag

SEQ ID NO: 3 - DNA, 20 bp, Artificial Sequence
  Description of Artificial Sequence Synthetic primer
ggaaacagrtgctaataccg

SEQ ID NO: 4 - DNA, 64 bp, Artificial Sequence
  Description of Artificial Sequence Synthetic primer
cgccgggggcgcgccccgggcggggcgggggcacggggggatcgtattaccgcggctgctggca

SEQ ID NO: 5 - DNA, 17 bp, Artificial Sequence
  Description of Artificial Sequence Synthetic primer
caccgctacacatggag

SEQ ID NO: 6 - DNA, 17 bp, Artificial Sequence
  Description of Artificial Sequence Synthetic primer
cggtgtgtacaagaccc

SEQ ID NO: 7 - DNA, 17 bp, Artificial Sequence
  Description of Artificial Sequence Synthetic primer
aacgcgaagaaccttac

SEQ ID NO: 8 - DNA, 18 bp, Artificial Sequence
  Description of Artificial Sequence Synthetic primer
ggcactaaagttggttca

SEQ ID NO: 9 - DNA, 19 bp, Artificial Sequence
  Description of Artificial Sequence Synthetic primer
cacccttgagttcagaatt

SEQ ID NO: 10 - DNA, 20 bp, Artificial Sequence
  Description of Artificial Sequence Synthetic primer
cggtattagcacctgtttcc

SEQ ID NO: 11 - DNA, 18 bp, Artificial Sequence
  Description of Artificial Sequence Synthetic primer
acttcggtaatgacgttg

SEQ ID NO: 12 - DNA, 20 bp, Artificial Sequence
  Description of Artificial Sequence Synthetic primer
ttctgcctttttgggatcaa

SEQ ID NO: 13 - DNA, 22 bp, Artificial Sequence
  Description of Artificial Sequence Synthetic primer
ccttgtttattcaagtgggtga

SEQ ID NO: 14 - DNA, 19 bp, Artificial Sequence
  Description of Artificial Sequence Synthetic primer
ggttaccttgttacgactt

(19) United States
(12) Patent Application Publication
(10) Pub. No.: US 2009/0004148 A1
(43) Pub. Date: Jan. 1, 2009

(54) Novel Probiotic

(75) Inventors: Sergey Rumenov Kostantinov, Amsterdam (NL); Hauke Smidt, Wageningen (NL); Paolo Bosi, Reggio Emilia (IT); Willem Meindert De Vos, Bennekom (NL)

Correspondence Address: FOLEY AND LARDNER LLP;SUITE 500, 3000 K STREET NW, WASHINGTON, DC, 20007, US

(73) Assignee: Wageningen Universiteit

(21) Appl. No.: 11/910,416

(22) PCT Filed: Mar. 30, 2006
(86) PCT No.: PCT/NL06/50067

§ 371 Date: Mar. 31, 2008

(30) Foreign Application Priority Data
Mar. 31, 2005 (EP) 05075747.5

(51) Int. Cl. A61K 35/74; C12N 1/20; C12Q 1/68; A61P 1/14

(52) U.S. Cl. 424/93.4; 435/252.1; 435/6

(57) ABSTRACT

The present invention relates to a novel Gram positive species that has been isolated from the porcine intestine. The Gram positive bacterium of the invention has unprecedented properties that relate to its capacity to grow on a wide range of industrial feeds including oligosaccharide carbohydrates both in vitro and in vivo in animals. The Gram positive bacterium of the invention may be used alone as probiotic or in synbiotic combination with oligosaccharide carbohydrates. Specific applications include the promotion of health, growth performance and colonization resistance in monogastric animals.

### FIELD OF THE INVENTION

The present invention relates to a Gram positive bacterium. In particular, it relates to a Gram positive bacterium which can be used as a probiotic.

### BACKGROUND OF THE INVENTION

Addition of probiotics to the diet, defined as live microbial feed supplements (Fuller, R. 1989. Probiotics in man and animals. Journal of Applied Bacteriology 66:365-378), is an approach, alternative to antibiotics, for the prevention and treatment of some infectious intestinal diseases in humans and animals. Such dietary intervention is partly based on the concept that specific strains selected from the healthy gut microbiota may have powerful anti-pathogenic and anti-inflammatory properties, and therefore may provide resistance to intestinal diseases (Isolauri et al. 2002. Probiotics: a role in the treatment of intestinal infection and inflammation? Gut 50:54-59). In monogastric animals, commensal microbiota contributes to intestinal protection against pathogens by competition for nutrients and pathogen binding sites, and/or regulation of immune response. The host resistance to infection might also be improved by the activities of the commensal bacterial such as the production of antimicrobial substances or the generation of restrictive physiological conditions, including acidification, caused by lactic acid and other fermentations (Teitelbaum et al. 2002. Annual Reviews Nutrition 22:107-138).

A critical phase in the development of farm animals is the transition to solid feed, also known as weaning, when a rapid proliferation of pathogens may occur.

The changes in the composition and activity of the small intestinal microbial community after weaning may be among the factors that predispose the animals to pathogenic infections. A significant decrease in the number of lactobacilli and an increase of coliforms has consistently been observed in the gastro-intestinal (GI) tract of piglets during the first weeks after weaning. In the immediate post-weaning period, the balance between the development of so called healthy commensal microbiota or the establishment of a bacterial intestinal disease can be easily tipped toward disease expression ((Zimmermann et al. 2001. Journal of Animal and Feed Sciences. 10(1):47-56)). Bacteria that were associated with diarrhoeal disease after weaning include enterotoxigenic Escherichia coli K88 (ETEC) and other E. coli strains (post-weaning colibacillosis), and Salmonella spp. (Hopwood et al. 2003. In J. R. Pluske, J. L. Dividich, and M. W. A. Verstegen (ed.), Weaning the pig. p. 199-219. Wageningen Academic Publishers, Wageningen, The Netherlands.

Thus far, no effective vaccines are available to control post-weaning colibacillosis, and many pathogenic E. coli strains show resistance to multiple antibiotics (Amezcua et al. 2002. Canadian Journal of Veterinary Research 66:73-78.). Hence, exogenous supplementation of lactobacilli has been reported among the possible alternatives to antibiotics for the reduction of post-weaning diarrhoea in weaning animals (Zimmermann et al. 2001. Journal of Animal and Feed Sciences. 10(1):47-56).

Konstantinov et al. 2004, Applied and Environmental Microbiology. 70(7): 3821-3830, described that a diet containing four fermentable carbohydrates fed to weaning piglets increased the endogenous lactobacilli diversity in the colon.

It is an object of the present invention to provide novel isolated bacteria having probiotic properties and compositions comprising these.

### DETAILED DESCRIPTION

The present invention relates in one aspect to an isolated Gram-positive bacterium comprising 16S ribosomal RNA (rRNA) which hybridises under stringent conditions to the nucleotide sequence

| | |
|---|---|
| 5′-CGC TTT CCC AAC GTC ATT-3′. | (SEQ ID NO. 1) |

and the genomic DNA of the bacterium shows more than 50%, more preferably at least 75%, or more, homology (as can be determined by DNA-DNA hybridization) to the genomic DNA of the Gram-positive bacterium which is deposited as CBS 117345.

The bacterium according to the invention has unprecedented properties that relate to its capacity to grow on a wide range of industrial feeds, which includes cheap and abundantly available oligosaccharide carbohydrates. It will grow on these substrates both in vivo, e.g. in an animal's intestine, and in vitro.

### Bacteria According to the Invention

The Gram-positive bacteria according to the invention comprise 16S rRNA, which will hybridise to SEQ ID NO. 1 under stringent conditions. In this context, “stringent conditions” refers to hybridisation conditions that allow for hybridization of sequences matching 100% with SEQ ID NO.1, while sequences will not hybridize that have one or more mismatches with SEQ ID NO.1. This is realized for example by hybridization at a constant temperature of 50° C. for 16 h and 0% (vol/vol) formamide in the hybridization buffer (0.9 M NaCl, 20 mM Tris-HCl [pH 7.5], 0.1% [wt/vol] sodium dodecyl sulfate) as described by (Konstantinov et al. 2004. Applied and Environmental Microbiology. 70(7):3821-3830). The 16S rRNA comprises a sequence, which matches 100% with SEQ ID NO.1. This will be the case when the 16S rRNA comprises a sequence, which is reverse complementary to the sequence of SEQ ID No. 1.

Members of the new species show high sequence similarity in almost-complete 16S rRNA gene sequencing, i.e. they show more than 90% similarity, preferably, more than 92%, 93%, 94%, 95%, 96%, 97% or 98%. Most preferably, they show more than 99.0%, 99.2%, 99.4%, 99.6% or 99.8% similarity. Similarity at the rRNA level can be determined by well-known programs for sequence comparison, such as ARB, check Probe and BLAST Programs. Based on 16S rRNA sequence identity, Lactobacillus kitasatonis (99%), L. crispatus (98%), and L. amylovorus (97%) were the nearest relatives of the new isolates, but their genomic DNA relatedness was found to be lower than 49%.

The genomic DNA of the bacterium of the invention shows at least 60%, 65%, 70%, 75%, 80%, 85%, 90%, 92% or 94% homology (herein also referred to as DNA-DNA re-association or DNA-DNA hybridization) to the isolated Gram-positive bacterium, which is deposited under accession number CBS 117345. In one embodiment, the genomic DNA of the bacterium of the invention shows at least 95%, 96%, 97%, 98% or 99% homology to the isolated Gram-positive bacterium, which is deposited under accession number CBS 117345. When reference is made herein to the bacterium deposited under CBS 117345, it is understood that derivatives thereof are encompassed herein, i.e. bacteria derived from the deposited bacterium by cell-division (e.g. growth in vitro or in vivo) and which retain the characteristics of strain CBS 117345.

Genomic DNA homology was established using the filter hybridization method described by Klijn et al. (Klijn et al. 1994. Systematic and Applied Microbiology. 17:249-256), except that for the nick translation [α-³²P] dCTP was used, and the hybridization temperature was 59° C. Thus, whether or not an isolated bacterium shows the above percentage of genomic DNA-DNA hybridization (or re-association) to the genomic DNA of the bacterium deposited at the CBS (Centraalbureau voor Schimmelcultures, Uppsalalaan 8, 3584 CT Utrecht, Netherlands) under Accession number CBS 117345 can, for example, be determined by labelling total genomic DNA using nick-translation and a radioactively labelled nucleotide (e.g. dATP or dCTP) and hybridizing the labelled DNA to a filter comprising total genomic DNA of the bacterium against which homology is to be compared. The radioactivity of bound DNA is then determined and the percentage of binding relative to the signal found in homologous hybridizations (e.g. CBS 117345-CBS 117345 hybridization) is then determined. See Examples and Klijn et al. (1994, supra, page 250, RH Column, last paragraph—page 251, LH Column, lines 1-4, incorporated herein by reference).

The Gram positive bacteria of the present invention have a genomic DNA G+C content of 35-36 mol %.

Six members of the new species were isolated and characterized (see Examples). Members of the new species appear as non-motile, non-spore-forming rods. These rods may be at least approximately 0.6 micrometer in width, and at least approximately 2 micrometer in length. They may occur, singly, in pairs or in long chains. In one embodiment, they are not wider than approximately 1 micrometer and not longer than approximately 20 micrometer. In another embodiment, colonies are at least 1.4 mm in diameter, circular to slightly irregular to rough in form and white.

All of the above characteristics in size shape, arrangement, Gram-stain and colonial appearance were determined by using cells grown on MRS agar plates for two days at 37° C.

There is no growth at 15° C., but the Gram positive bacterium of the invention grows at 45° C. The organism is facultatively anaerobic and produces D- and L-lactic acid homofermentatively. Catalase is not produced. Acid is produced without gas formation from D-glucose, D-mannose, maltose, galactose, D-fructose, lactose, esculin, sucrose, amidon, mannitol, cellobiose, salicin, trehalose, amygdalin, N-acetylglucosamine, arbutin, ribose, glycogen, 5 keto-gluconate.

There is no acid formation from glycerol, erythritol, D-arabinose, L-arabinose, D-xylose, L-xylose, melezitose, rhamnose, adonitol, β methyl-xyloside, sorbitol, L-sorbose, dulcitol, inositol, α methyl-D-mannoside, α methyl-D-glucoside, arbutin, inulin, xylitol, D-turanose, D-lyxose, D-tagatose, D-fucose, L-fucose, D-arabitol, L-arabitol, gluconate, 2 keto-gluconate.

They are capable of degrading oligosaccharide carbohydrates such as fructooligosaccharides (FOS) and sugar beet pulp (SBP), which are cheap and readily available waste stream products from the agricultural industry. Their ability to degrade FOS and SBP may lead to their selective enrichment by FOS and SBP in the intestine of an animal. The species was isolated for the first time from the porcine intestine before weaning as described in the examples.

### Compositions and Uses of the Invention

In another aspect, the present invention provides a composition for use in the food, feed or breeding industry. A composition according to the invention comprises at least one isolated Gram-positive bacterium according to the invention, but may also comprise two or more of the bacteria according to the invention (mixtures). Preferably the bacteria are live or viable, although in one embodiment also dead/non-viable bacteria may be used. When dead or non-viable bacteria are used, the dosage (colony forming units, cfu) can be determined prior to killing of the bacteria. The composition may have any form, it may suitably be formulated as a solid, a liquid or a semi-liquid composition. For instance, it may be in freeze dried form, e.g. in a capsule. If it is in a freeze dried form it is preferably added to water or another liquid for use.

It may be added to the food or feed shortly before use or it may already be included in the feed or food before it reaches the end user. It may for instance be added to liquid feed, or dairy products, such as milk, yoghurt, desserts, spreads, butter and cheese.

A culture of the bacterium of the invention may be used alone as probiotic, or in synbiotic combination with oligosaccharide carbohydrates. Therefore, a feed or food composition further comprising oligosaccharide carbohydrates is also encompassed by the present invention. Suitable oligosaccharide carbohydrates include fructoooligosaccharides and sugar beet pulp, but also other carbohydrates as described herein, such as D-raffinose, sucrose, lactose, etc.

In one embodiment, the composition is for use in the breeding industry, e.g. for weaning young animals. In those cases it may be used to counteract or compensate for the stress effects usually experienced by weaned animals, such as a reduced growth rate, which accompany weaning. Therefore, in yet another aspect, the invention provides a method for weaning an animal by administering a composition to the invention to a young animal before weaning, in particular a young farm animal e.g. to a piglet, or a pet, such as a puppy or a kitten.

Of particular interest is the use of the bacterium of the present invention as a probiotic. The bacterium of the present invention as such or in combination with oligosaccharide carbohydrates, may be used to promote health, growth performance and colonisation resistance, in particular in monogastric animals, including human beings. Preferably, it is used to inhibit or reduce the growth of pathogenic organisms, such as enterotoxigenic E. coli, and/or it is used to reduce or prevent diarrhoea. The method comprises the administration of a composition according to the invention to the animal. In this context, “monogastric animal” refers to any animal with one stomach, which applies to most carnivores and omnivores, with the exception of ruminants.

Suitable amounts of one or more bacteria may be administered as such or as part of a composition (food or feed) on a daily (once or several times per day), weekly or monthly basis. A suitable dosage of the bacterium, or of each bacterium when a mixture is used, can be determined by the skilled person. Suitable dosages include preferably at least 1×10⁶ cfu, preferably between about 1×10⁶-1×10¹² cfu (colony forming units) per day per strain, more preferably between about 1×10⁷-1×10¹¹ cfu/day, more preferably about 1×10⁸-5×10¹⁰ cfu/day, most preferably between 1×10⁹-2×10¹⁰ cfu/day.

The compositions according to the invention may comprise other components, such as other probiotic bacteria, prebiotics, minerals, nutrients, flavourings, fat, protein, carbohydrates, etc.

### Methods of the Invention

In another aspect, the invention provides a method for detecting an isolated Gram-positive bacterium according to the invention in a sample. The method comprises:
providing a probe which includes a marker and a nucleotide sequence according to SEQ ID No.1;
contacting the probe with the sample under hybridising conditions;
removing unbound probe; and
detecting the marker indicating bound probe;

A sample may be obtained from any excrement, which is likely to reflect the contents of the intestines. A sample may suitably be obtained from faeces. A more invasive method is sampling by endoscopy.

Using the method of the invention, an indication may be obtained whether colonisation has occurred, in particular if it is combined with quantification of the bacteria. The method may also be used to get an indication of the health of the animal from which the sample has been obtained. In another embodiment of the invention, the method is used to screen for related organisms from other sources than or in porcine intestine.

Yet another aspect of the invention is a method for inhibiting or preventing the growth of intestinal pathogens by administering a Gram-positive bacterium or a composition according to the invention to an animal. In pigs or humans, the method of the invention may be used to inhibit or prevent the growth of porcine or human intestinal pathogens, e.g. Clostridium, Salmonella, Shigella, Yersinia. Alternatively, the method may be used to inhibit or prevent the growth of other bacteria which are normal colonists of the mammalian gastrointestinal tract, e.g. Escherichia, Enterobacter, Klebsiella, but which may occasionally be associated with diseases of pigs or humans. In one embodiment, the method of the invention is used to inhibit or prevent the growth of enterotoxigenic Escherichia coli (ETEC). In a preferred embodiment, the animal is a pet, such as a cat, a hamster or a dog or a human being.

### SHORT DESCRIPTION OF THE FIGURES

FIG. 1. Growth of strain OTU171_{—}001^{T} in MRS broth containing either 1% FOS, 1% D-glucose, 1% D-fructose or 1% SBP.

FIG. 2. Phylogenetic analysis of strain OTU171_{—}001^{T} (L. sobrius) and selected members of L. delbrueckii 16S rRNA gene cluster of the genus Lactobacillus. The tree was calculated using the neighbour-joining method with partial 16S rRNA gene sequences (E. coli positions 107-1433), using the ARB software package (Ludwig et al., 2004 Nucl Acids Res 32, 1363-1371). The bar represents 5% sequence divergence.

FIG. 3. Effect of dietary supplementation with L. sobrius strain 001^{T} on total IgA in saliva, blood serum and jejunum secretions of ETEC challenged pigs (least squares means±SEM).

# Effect of diet: p=0.10.

*Effect of diet: p<0.05.

FIG. 4. Effect of dietary supplementation with L. sobrius strain 001^{T} on sIgA against L. sobrius strain 001^{T} in saliva, blood serum and jejunum secretions of E. coli challenged pigs (least squares means±SEM).

*Effect of diet: p<0.05

### EXAMPLES

### Materials and Methods

Strains were tested for carbohydrate fermentation abilities using the API 50 CHL system (bioMérieux). In addition, the degradation of fructooligosaccharides (FOS) and sugar beet pulp (SBP) by L. sobrius_{—}001^{T} was tested using MRS as basal media (without carbohydrates) as described by Barrangou et al., 2003 (PNAS USA 100: 8957-8962). The bacterial culture was propagated at 37° C., aerobically in MRS basal media consisting of: 1% bactopeptone (wt/vol), 0.5% yeast extract (wt/vol), 0.2% dipotassium phosphate (wt/vol), 0.5% sodium acetate (wt/vol), 0.2% ammonium citrate (wt/vol), 0.02% magnesium sulfate (wt/vol), 0.005% manganese sulfate (wt/vol), 0.1% Tween 80 (vol/vol), 0.003% bromocresol purple (vol/vol). The MRS basal medium was autoclaved, and after filter sterilization either D-glucose (dextrose), D-fructose, or FOS (Raftilose P95, Orafti) were added to a concentration 1% sugar (wt/vol). In the case of SBP degradation, the basal media was supplied with 1% SBP (wt/vol) and then boiled for 20 min in a water bath (100° C.).

Cell shape, size and arrangement, Gram-stain and colonial appearance were determined by using cells grown on MRS agar plates for 2 days at 37° C. Production of gas from glucose was also examined. Catalase formation, and growth at 15 and 45° C. were done using MRS broth as the basal medium.

Almost-Complete 16S rRNA Gene Sequences

(approximately 1.5 Kb) were determined for the six representative strains. The 16S rRNA gene (corresponding to Escherichia coli positions 8-1492) was amplified by PCR, using primers S-D-Bact-0011-a-A-17 (AGA GTT TGA T(C/T)(A/C) TGG CTC AG), and S-D-Bact-1492-a-A-19 (GGT TAC CTT GTT ACG ACT T) (Leser et al., 2002 Appl Environ Microbiol 68, 673-690) and the product was further cloned and sequenced as described (Konstantinov et al., 2004 Appl. Environ. Microbiol. 70(7):3821-3830). The sequence of OTU_{—}001^{T} was deposed under the accession number AY700063 in the GenBank. Phylogenetic analysis was performed using the ARB software package (Ludwig et al., 2004 Nucl Acids Res 32, 1363-1371). In order to estimate the approximate genome size of isolates and to analyze their genomic diversity, pulsed-field gel electrophoresis (PFGE) of Apa I digested chromosomal DNA was performed as described previously (McCartney et al., 1996 Appl Environ Microbiol 62, 4608-4613). The genome size of the isolates was calculated according to the λ PFG standard ladder using the program Quantity One (Bio-Rad). G+C content was calculated based on the determination of genomic DNA Tm (Marmur et al., 1962 J Mol Biol 4, 109-118). DNA-DNA relatedness was analyzed by filter hybridization according to (Klijn et al., 1994 System Appl Microbiol 17, 249-256), except that for the nick translation [α-³²P] dCTP was used, and the hybridization temperature was 59° C. (±25° C. below Tm). Extraction of whole-cell proteins and their separation by SDS-PAGE was done using standard protocols (Maniatis et al., 2003 Molecular Cloning: a Laboratory Manual, 2nd ed. Cold Spring Harbor Laboratory Press, Cold Spring Harbor, N.Y.), and SDS-PAGE protein fingerprints were compared using the Bionumerics software package version 3.0. (Applied Maths).

### Feed Experiments.

The animal experiment consisted of 3 replications. Each replication was done using 16 weaned piglets (21 days of age) assigned to one of two dietary treatments, a basic diet (control diet) and a basic diet containing L. sobrius strain 001^{T} (LAB diet). The piglets were balanced for litter and weight, and sex was registered. Piglets were fed a standard diet, containing 4% dried sugar beet pulp. The LAB diet contained in addition to the basal diet 1 ml/day of a skimmed milk solution supplemented with 10¹⁰ CFU of L. sobrius strain 001^{T}, while 1 ml milk only was added to the control diet. The experimental period was divided in two phases: an adaptation phase, when subjects were adjusted to the experimental diets (day 0 till day 7), and a challenge period (day 7 till day 14). The skimmed milk with and without L. sobrius strain 001^{T} was supplied orally by a sterile syringe on day 0 and day 1, and then till the end of the experiment in the trough, carefully mixed with the feed every morning. Pigs were penned on a mesh floor, in groups of four on day 0 and day 1, and then individually. On day 7, the piglets were challenged with 1.5 ml of a suspension containing 10¹⁰ CFU/ml enterotoxigenic E. coli K88 (ETEC).

### Diarrhoea Score and ETEC Quantification.

Diarrhoea scores were recorded by visual appraisal of each subject using a 5-point scoring system (1 to 5), the excrements being hard (score 1) and watery fecal excrements (score 5). Two and three days after the challenge, individual fecal samples were collected, and the quantification of ETEC and of total E. coli excretion, was done as reported previously Bosi, et al. 2004. J. Anim. Sci. 52.

Four random samples per group per replication (in total 12 samples per diet) were used for further isolation of L. sobrius strains and their taxonomic characterization as described in Konstantinov, et al, 2004 vide supra.

Total Secretory IgA(sIgA), and Anti-L. sobrius Strain 001^{T} and ETEC Specific sIgA Titres.

At the day of challenge, saliva and blood samples were collected from all subjects. Saliva samples were collected again one day before the sacrifice, while blood samples were collected immediately before sacrifice. All sIgA determinations were done by ELISA. For total sIgA detection, 96-well microliter plates were coated with Goat anti-pig sIgA, affinity purified (BETHYL Laboratories, Montgomery, Tex.) and diluted in carbonate-bicarbonate buffer, 50 mM, pH 9.4. Subsequently, phosphate-buffered saline (PBS) supplemented with 0.2% (v/v) Tween 20 was added to the wells to block the remaining binding sites. Pig Immunoglobulin Reference Serum (BETHYL, Laboratories, Montgomery, Tex.) was used as specific antibody for standard curve, Goat anti-Pig sIgA-HRP conjugate (BETHYL Laboratories, Montgomery, Tex.) as secondary antibody and ABTS (ROCHE Diagnostics) as enzyme substrate. Absorption was read at 405 nm by a microplate reader (Sunrise Microplate Reader, TECAN ITALIA). The concentration values was expressed as μg/ml. L. sobrius strain 001^{T} and ETEC-specific sIgA titres were determined according to Ibnou-Zekr et al. 2003. Infect Immun. 71(428-346) and Van den Broeck et al. 1999. Infect. Immun. 67:520-526, respectively.

### Collection of Samples at Sacrifice.

On days 13 or 14, all the piglets, equally distributed by treatment and selected on a random basis within each treatment, were anaesthetized with sodium thiopenthal (10 mg/kg body weight). Euthanasia was performed by intracardiac injection of Tanax® (0.5 ml/kg body weight; Intervet Italia, Peschiera Borromeo, Italy).

The small intestine was sampled at ¼ and ¾ of its total length for morphometric analysis of villi and crypts, and measured as reported Bosi et al 2002, vide supra). For the determination of sIgA, a 50 cm segment obtained from the final jejunum was immediately processed as described Evans et al. 1980. Scand. J. Immunol. 11:419-429. Samples of gut content were also collected from stomach, duodenum, final jejunum, cecum and colon for pH measure. Lumen samples collected from the terminal ileum were divided into aliquots that were used for genomic DNA extraction by using the Fast DNA Spin Kit (Qbiogene, Inc, Carlsbad, Calif.) and fixed for fluorescent in situ hybridization (FISH), respectively. A DNA oligonucleotide probe L-*-OTU171-0088-a-A-18 (Table 2) targeting the 16S rRNA of L. sobrius sp. nov. was used for FISH analysis of ileal lumen samples as reported (Konstantinov et al. 2004, vide supra).

### Statistical Analysis.

Data were analyzed by analysis of variance using the GLM procedure (SAS version 8.1, SAS Institute, Cary, N.C.) with a 3-factor design, including diet, batch, sensitivity of intestinal villous to ETEC adhesion, and 1^{st} level interactions.

### Lactobacillus-Specific PCR Amplification.

The PCR approach was applied as previously described (Konstantinov et al. 2004, vide supra) and the primers used in this study are listed in Table 2. PCR was performed using the Taq DNA polymerase kit from Life Technologies (Gaithersburg, Md.). PCR mixtures (50 μl) contained 1.25 U μl of Taq polymerase (1.25 U), 20 mM Tris-HCl (pH 8.5), 50 mM KCl, 3.0 mM MgCl₂, 200 μM of each dNTP, 100 nM of the primers S-D-Bact-0011-a-A-17 and S-G-Lab-0677-a-A-17 and 1 μl of DNA diluted to approximately 1 ng/μl and UV sterilized water (mili Q). The samples were amplified in a thermocycler T1 Whatman Biometra (Göttingen, Germany) and the cycling consisted of pre-denaturation at 94° C. for 5 min; 35 cycles of 94° C. for 30 sec, 66° C. for 20 sec, and 68° C. for 40 sec, and a final extension at 68° C. for 7 min. PCR products were then used as templates in nested PCR reactions, using: S-G-Lab-0159-a-S-20 and S-*-Univ-0515-a-A-GC and following the cycling programme of 94° C. for 5 min, and 35 cycles of 94° C. for 30 sec, 56° C. for 20 sec, 68° C. for 40 sec, and 68° C. for 7 min final extension. Aliquots of 5 μl were analyzed by electrophoresis on 1.2% agarose gel (w/v) containing ethidium bromide to check for product size and yield.

### Denaturing Gradient Gel Electrophoresis (DGGE) Analysis.

The amplicons obtained from the lumen-extracted DNA were separated by DGGE (Dcode™ system; Bio-Rad Laboratories, Hercules, Calif.) using a previously described protocol (Konstantinov et al. 2004, vide supra). In brief, electrophoresis was performed in an 8% polyacrylamide gel of 37.5:1 acrylamide-bisacrylamide (dimensions 200×200×1 mm) with a gradient of 30-60% for the separation of PCR products obtained with primers S-G-Lab-0159-a-S-20 and S-*-Univ-0515-a-A-GC. After a short run at 200 V for 5 min, the gels were electrophoresed for 16 h at 85 V in 0.5×TAE buffer at a constant temperature of 60° C. and subsequently stained with AgNO₃.

### Reference Strains Used for Real-Time PCR Analysis.

L. sobrius strain 001^{T} was propagated at 37° C., anaerobically in deMan, Rogosa, Sharpe (MRS) broth (Difco, Le Point de Claix, France), while ETEC was grown in Luria-Bertani broth containing 1% tryptone, 0.5% yeast extract and 1% NaCl, pH 7.0. Both cultures (2 ml) were harvested after 24 h by centrifugation at 5,000×g for 10 min and washed with 0.2 μm pore size filtered PBS (per liter: 8 g NaCl, 0.2 g KCl, 1.44 g Na₂HPO₄ and 0.24 g KH₂PO₄; pH 7.2). The bacterial pellet was finally resuspended in 1 ml PBS. Ten μl of each culture was used for total cell counts determination based on 4′, 6-diamino-2-phenylindole (DAPI) staining coupled to microscopy analysis as described Konstantinov et al., vide supra). Isolation of genomic DNA from the remaining 990 μl bacterial culture was done using the Fast DNA Spin Kit (Qbiogene). Finally, L. sobrius and ETEC genomic DNA were diluted to concentrations ranging from 10⁸ to 10 cells/ml per real-time PCR reaction and used for generation of real-time PCR standard curves.

Real-Time PCR Assay for Quantification of L. sobrius Strain 001^{T} and ETEC.

Strain- and species-specific quantitative PCR detection targeting L. sobrius strain 001^{T} were achieved using primers OTU171_RDA_F and OTU171_RDA_R, and L-*-OTU171-0077-a-S-2 S-G-Lab-0159-a-A-2, respectively (Table 2). For ETEC quantification, primers K88AD_F and K88AD_R were employed (Table 2). In addition, 16S rRNA gene-targeted primers, 968F and R1401 (Table 2) were applied for total bacterial quantification. Real-time PCR was performed on an iCycler IQ real-time detection system associated with the iCycler optical system interface software version 2.3 (Bio-Rad, Veenendaal, The Netherlands). A reaction mixture (25 μl) consisted of 12.5 μl of IQ SYBR Green Supermix (Bio-Rad), 0.2 μM of each primer set, and 5 μl of the template DNA. The PCR conditions for the specific L. sobrius strain 001^{T} quantification were: an initial DNA denaturation step at 95° C. for 3 min, followed by 40 cycles of denaturation at 95° C. for 15 sec, primer annealing and extension at 60.3° C. for 45 sec. The same conditions were used for the species-specific quantification of L. sobrius except that the primer annealing and extension was 62.5° C. For total bacterial quantification, conditions were 94° C. for 5 min, and 35 cycles of 94° C. for 30 sec, 56° C. for 20 sec, 68° C. for 40 sec (Nübel et al. 1996. Journal of Bacteriology 178:5636-5643). For the detection of ETEC, conditions were: 40 cycles of 92° C. for 45 sec, 50° C. for 45 sec, 72° C. for 45 sec (Alexa et al. 2001. Veterenary Medicine 46:46-49). For the determination of the number of L. sobrius strain 001^{T} and ETEC present in each sample, fluorescent signals detected from two serial dilutions were compared to a standard curve generated with the respective bacterium in the same experiment. Serially diluted genomic DNA of L. sobrius strain 001^{T} was used as real-time PCR control for total bacteria quantification.

### Example 1

### Isolation of the Bacteria of the Invention

A DNA oligonucleotide probe L-*-OTU171-0088-a-A-18 (5′-CGC TTT CCC AAC GTC ATT-3′) (Konstantinov et al. 2004 vide supra) targeting the 16S rRNA of L. amylovorus-like phylotype OTU171 was used for screening of a range of Lactobacillus isolates from piglets (21 days of age) housed at different locations. In total 192 isolates grown on Lactobacillus selective agar MRS (Difco, Le Point de Claix, France) were screened by fluorescence in situ hybridization (FISH) using the CY3-labelled phylotype specific probe in combination with image analysis as described (Konstantinov et al. 2004 vide supra). Two Lactobacillus amylovorus-like strains were identified in the feces of piglets housed on a farm near Wageningen, the Netherlands and indicated further as OTU171_{—}001^{T} and OTU171_{—}002 in this study. Three strains (OTU171_{—}003, OTU171_{—}004, OTU171_{—}005) were isolated from the feces of piglets kept on a farm near Bologna, Italy, and one isolate was found in the ileal lumen sample of a piglet reared on a farm near Bristol, UK (OTU171_{—}006). The six isolates were selected for further characterization by phenotypic and molecular taxonomic methods. L. acidophilus DSMZ20079^{T}, L. amylovorus DSMZ 20531^{T}, L. crispatus DSMZ 20584^{T}, L. gallinarum DSMZ 10532^{T}, L. helveticus DSMZ 20075^{T}, and L. kitasatonis JCM1039^{T} were used as reference strains. All further cultivation of Lactobacillus isolates and reference strains was anaerobically on MRS agar or in MRS broth at 37° C.

### Example 2

### Characterisation of the Strain

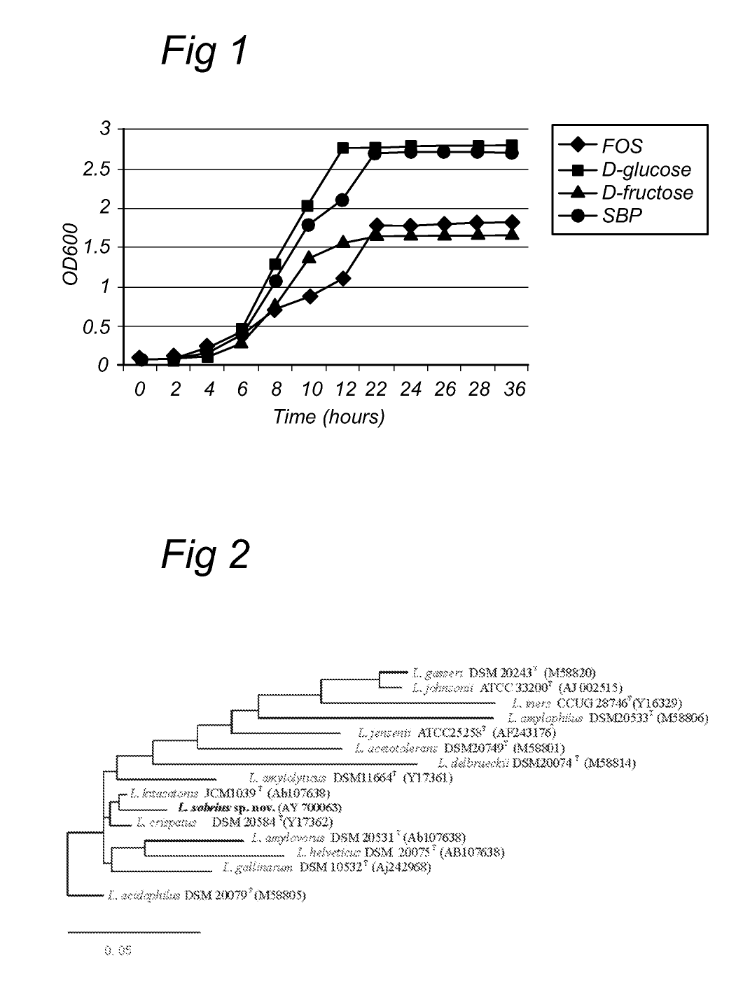

The L. amylovorus-like isolates hybridizing to the OTU171 probe were Gram-positive, non-spore-forming and non-motile rods and colonies of these strains were white with circular to irregular shapes. All strains displayed similar fermentation characteristics, as measured by API 50 CHL. Phenotypic characteristics that differentiate the strains from other reference strains are summarized in Table 1. In contrast to L. kitasatonis, all new isolates were able to ferment D-raffinose. Strain OTU171_{—}001^{T} was also tested for ability to ferment FOS and SBP (FIG. 1). The strain grew in MRS-FOS to a final cell density OD600=1.81. This MRS-FOS growth was slower compared to MRS-D-glucose, but similar to the cell density after strain inoculation on MRS-D-fructose (FIG. 1). A positive growth, with the approximately the same rate as the growth on MRS-D-glucose, was obtained on MRS-SBP broth as well (OD600=2.7). Without carbohydrate supplementation, the bacterial growth in the semisynthetic medium was unable to sustain above OD600 nm=0.25.

The strains produced DL-lactic acid, did not grow at 15° C., but did grow at 45° C., were catalase-negative. Acid is produced without gas formation from D-glucose, D-mannose, maltose, galactose, D-fructose, lactose, esculin, sucrose, amidon, mannitol (2 of 6 strains, including strain OTU171_{—}001^{T}), cellobiose (3 out of 6 strains, including strain OTU171_{—}001^{T}), salicin (2 out of 6 strains, including strain OTU171_{—}001^{T}), trehalose (2 out of 6 strains, including strain OTU171_{—}001^{T}), amygdalin (2 out of 6 strains, including strain OTU171_{—}001^{T}, weak reaction), N-acetylglucosamine (2 out of 6 strains, including strain OTU171_{—}001^{T}), arbutin (2 out of 6, including strain OTU171_{—}001^{T}), ribose (2 out of 6 strains, including strain OTU171_{—}001^{T}, weak reaction), glycogen (3 out of 6 strains, fermented by strain OTU171_{—}001^{T}), 5 keto-gluconate (2 strains out of 6, fermented by strain OTU171_{—}001^{T}, weak reaction). FOS and SBP are also fermented by OTU171_{—}001^{T}. There is no acid formation from glycerol, erythritol, D-arabinose, L-arabinose, D-xylose, L-xylose, melezitose, rhamnose, adonitol, β methyl-xyloside, sorbitol, L-sorbose, dulcitol, inositol, α methyl-D-mannoside, α methyl-D-glucoside, arbutin, inulin, xylitol, D-turanose, D-lyxose, D-tagatose, D-fucose, L-fucose, D-arabitol, L-arabitol, gluconate, 2 ceto-gluconate.

The DNA G+C content of the six strains ranged from 35-36 mol % (Table 1).

Almost-complete 16S rRNA gene sequences of six representative isolates showed high sequence similarities (>99%). Subsequent phylogenetic analysis confirmed the association between the newly isolated strains and species that belong to the Lactobacillus delbrueckii group of the genus Lactobacillus (Collins et al. 1991; Mukai et al. 2003) (FIG. 2). High levels of sequence relatedness were found with L. kitasatonis (99%), L. crispatus (98%), L. amylovorus (97%) and L. gallinarum (97%).

The analysis of the isolates whole-cell proteins by SDS-PAGE displayed marked differences in comparison with those of the reference strains. An abundant 50 kD protein was detected in the SDS fingerprints of all new isolates. After cluster analysis of the SDS-PAGE protein profiles, the fingerprints of the new isolates formed a coherent cluster with a similarity higher than 85%, while they were only distinctly related to the examined type strains with a similarity index below 70%.

Furthermore, the new isolates were compared to the closest type strains by DNA-DNA hybridization. Labelled DNA of OTU171_{—}001^{T}, OTU171_{—}002 and OTU171_{—}003 reassociated at high level (78-100%) with unlabelled DNA from the six OTU171 strains, while only low levels of reassociation (2-49%) were observed with the examined closely related Lactobacillus species (Table 3). The results indicated that the isolates, analyzed during the course of the study, belong to a single species that differ from the closest type strains.

To further assess genomic diversity of the six isolates, the PFGE profiles of Apa I digested chromosomal DNA were visually compared. All isolates displayed distinct PFGE profiles from each other. The average genome size was 1.2 Mb.

The new species is further referred to as L. sobrius with the type strain OTU171_{—}001^{T}.

### Example 3

### Animal Observations

Piglets fed the LAB diet were found to grow significantly faster (+74%, P<0.05), while they did not show different feed intake, than the piglets fed on the control diet (Table 4). An increased number of days with diarrhea score >2 was observed in the piglets fed the LAB diet (+1.9 days, P<0.05), while the average diarrhea score was not significantly changed. Fecal excretion of total E. coli and ETEC was not affected on day 2 of the challenge period. On day 3 post-infection a higher fecal shedding of ETEC, however not statistically significant, was observed in the piglets fed the LAB diet (Table 4).

The pH in stomach, duodenum, jejunum, cecum and colon was not significantly different between the two dietary groups (Table 5). The morphology of small intestine, as measured by villous height and crypt depth, was also not modified by the diet.

### Example 4

### IgA and sIgA Titers

At the time of challenge (day 7), total contents of sIgA in saliva and blood serum in the piglets was not affected by the dietary addition of L. sobrius strain 001^{T} (FIG. 2). One week later, total content of sIgA in saliva was maintained in the piglets fed the LAB diet, while values decreased significantly in the control diet (p<0.05). For blood serum, total sIgA increased in piglets, but the L. sobrius strain 001^{T} supplemented group tended to have more sIgA, compared to the control (p=0.10). Total sIgA contents in jejunum secrete was not affected.

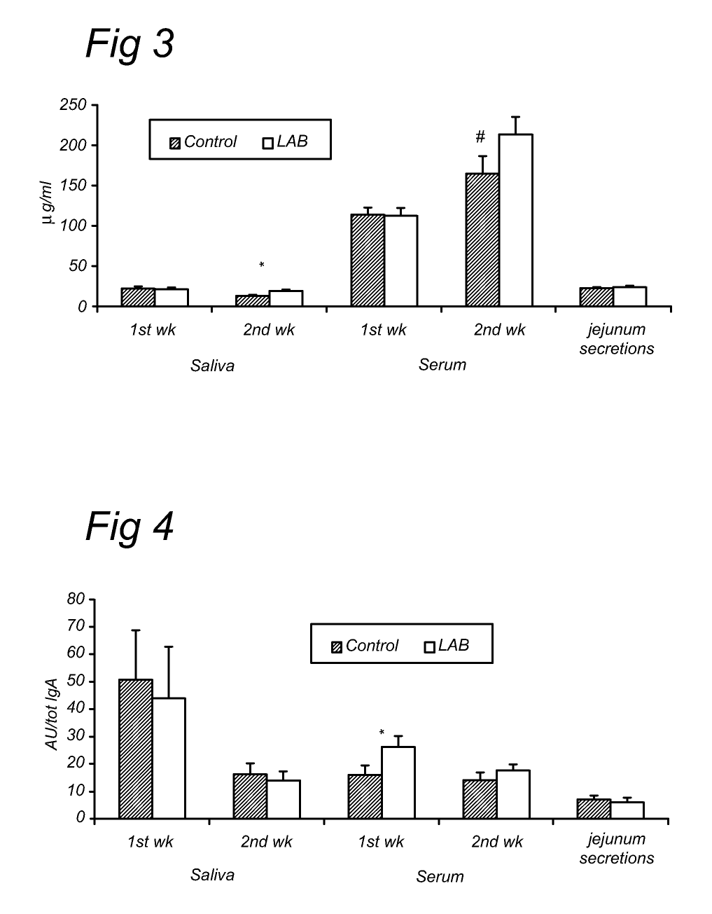

The effect of dietary supplementation with L. sobrius strain 001^{T} on sIgA against this strain in saliva, blood serum and jejunum secretions of ETEC challenged pigs is presented in FIG. 3. sIgA antibodies against L. sobrius strain 001^{T} were detected in nearly all pigs irrespective of the diet. Its concentration (expressed on total sIgA) was significantly higher in piglets fed the LAB diet only in the case of blood serum collected after 1 week of supplementation (p<0.05). In general, without considering the diet, sIgA against L. sobrius strain 001^{T} was still present after 2 weeks in intestinal secretion and a small reduction in blood was seen. However, a very strong reduction was observed for L. sobrius strain 001^{T} recognizing sIgA in saliva, changing from first week to second week sample. ETEC-specific sIgA's did not statistically vary between the LAB and control groups.

### Example 5

### Quantitative PCR Detection of L. sobrius Species in Culture Medium and in Ileal Lumen Samples

The number of L. sobrius strain 001^{T} as added to the LAB diet was quantified by real-time PCR using species-specific primers. There were no significant differences between the results obtained by the real-time PCR assay, direct FISH counting of the DAPI stained cells or viable counts when samples were spiked with various amounts (10⁹-10⁵ cells) of L. sobrius strain 001^{T}. Moreover, the real-time PCR efficiency was not affected when comparing DNA extracted from MRS grown cultures or ileal samples. Furthermore, real-time PCR analyses were performed to quantify L. sobrius species in ilea samples of four piglets received LAB diet, and the results were compared with those obtained by FISH. For three samples both methods, quantitative PCR and FISH, detected the targeted species at the same levels (approximately 10⁸/g). In the fourth sample, however, L. sobrius was assessed by quantitative PCR, but not FISH, due to the detection limit of the FISH technique (Table 6).

### Example 6

### Persistence of L. sobrius Strain 001^{T} and ETEC in Ilea Lumen Samples of Piglets Examined by Real-Time PCR

To evaluate whether the ETEC number in ileal lumen was affected in a quantitative manner by the addition of the L. sobrius strain 001^{T}, both species- (L. sobrius) and strain-specific (L. sobrius strain 001^{T} or ETEC) real-time PCR assays were performed. The counts were determined in the ileal samples and compared between the piglets fed the LAB and control diet (Table 7). The administered L. sobrius strain 001^{T} was detected (>10³ cells/g) in 12 out of 16 piglets fed the LAB diet with a population size of 0.66±1×10⁸ cells/g ileal lumen (mean count±SD). Within the LAB group, L. sobrius species was identified in significant numbers (>10³ cells/g) in 13 out of 16 samples, and the population of L. sobrius per gram of ileal lumen was 1.6±0.9×10⁸ cells. The species was detected at significantly lower level in the control group (2.3±3×10⁴; p<0.05). ETEC was detected in the ilea samples from 12 out of 16 piglets fed the control diet (>10³ cells/g) and was present in a relatively larger population (1±10⁶ cells/g). In contrast, the population of this pathogen was significantly lower in the LAB group (4.2±0.7×10⁴; p<0.05). Interestingly, ETEC was not detected in ileal samples of four piglets of LAB group harboring large populations (>10⁸ cells/g) of L. sobrius strain 001^{T}. The total number of bacteria was not significantly different between the two dietary groups (Table 7).

### Example 7

### DGGE Analysis of the Lactobacillus Community in the Ileum of Weaning Piglets

Complimentary to real-time PCR detection, Lactobacillus-specific 16S rRNA gene-targeted amplification, in combination with DGGE fingerprinting analysis, was performed for piglets' ileal lumen samples. While some individual variations were found both in the number and position of the Lactobacillus-specific amplicons, the fingerprints show high similarity. However, a market difference in the presence of a specific DGGE band outlining the administered L. sobrius strain 001^{T} was found in a majority (7 out of 8) of the ileal samples from piglets fed the LAB diet. The amplicon was observed with lower frequency and intensity in the ileal samples from piglets fed the control diet. These data were consistent with the real-time PCR results, and indicated higher emergence of L. sobrius in LAB compared to the control group after the experimental ETEC challenge.

TABLE 1
DNA G + C content and phenotypic characteristics of Lactobacillus
amylovorus-like strains and closely related lactobacilli.

| | Characteristics | | | | | | | | |
|---|---|---|---|---|---|---|---|---|---|
| | 1 | 2 | 3 | 4 | 5 | 6 | 7 | 8 | 9 |
| | DNA G + C content (mol %) | | | | | | | | |
| | 35- | 37- | 36- | 33- | 36- | 33- | 32- | 33- | 38- |
| | 36 | 40 | 38 | 36 | 37 | 34 | 40 | 35 | 40 |
| Growth at 15° C. | − | − | − | − | + | − | − | + | − |

Fermentation of:

| | | | | | | | | | |
|---|---|---|---|---|---|---|---|---|---|
| D− Raffinose | + | − | D | + | + | − | + | D | − |
| Sucrose | + | + | + | + | + | + | + | + | − |
| Lactose | + | DW | + | + | D | + | + | + | + |
| Mannitol | DW | DW | − | D | − | − | − | − | − |
| Cellobiose | D | D | D | + | + | + | + | + | − |
| Salicin | DW | D | + | + | + | + | + | + | − |
| Ribose | DW | − | − | D | − | − | − | − | − |
| Trehalose | D | D− | − | D | − | + | + | D | D |
| Melibiose | D | − | D | + | + | W | − | D | − |
| Amygdalin | DW | − | D | + | + | + | + | + | − |

Taxa: 1, L. amylovorus-like (OTU171) strains; 2, L. kitasatonis; 3, L. amylovorus; 4, L. crispatus; 5. L. gallinarum; 6, L. gasseri; 7, L. acidophilus; 8, L. johnsonii; 9, L. helveticus. Data about the taxas 2 to 9, belonging the L. acidophilus group, is from Mukai et al., 2003; +, Positive; D, strain-dependent; D−, usually negative; −, negative; W, weak reaction. All strains produce acid from D-glucose, galactose, D-fructose, D-mannose and maltose and produce DL-lactic acid. No strains produce acid from arabinose, xylose, rhamnose, melezitose or sorbitol.

TABLE 2
List of DNA oligonucleotides used in this study.

| Oligonucleotides | 5′-3′ | Target | Reference |
|---|---|---|---|
| L-*-OTU171-0088-a-A-18 | CGC TTT CCC AAC GTC ATT | 16S rRNA | |
| S-D-Bact-0011-a-A-17 | AGA GTT TGA T(C/T)(A/C) TGG CTC AG | 16S rRNA | (3) |
| S-G-Lab-0159-a-S-2 | GGA AAC AG(A/G) TGC TAA TAG CG | 16S rRNA | (2) |
| S-*-Univ-0515-a-A-24-GC | CGC CGG GGG CGC GCC CCG GGC GGG | 16S rRNA | (2) |
| | GCG GGG GCA CGG GGG G ATC GTA TTA | | |
| | CCG CGG CTG CTG GCA | | |
| S-G-Lab-0677-a-A-17 | CAC CGC TAC ACA TGG AG | 16S rRNA | (2) |
| R 1401 | CGG TGT GTA CAA GAC CC | 16S rRNA | (4) |
| F 968 | AAC GCG AAG AAC CTT AC | 16S rRNA | (4) |
| K88AD_F | GGC ACT AAA GTT GGT TCA | ETEC | (1) |
| K88AD_R | CAC CCT TGA GTT CAG AAT T | ETEC | (1) |
| S-G-Lab-0159-a-A-2 | CGG TAT TAG CAC CTG TTT CC | 16S rRNA | |
| L-*-OTU171-0077-a-S-2 | ACT TCG GTA ATG ACG TTG | 16S rRNA | |
| OTU171_RDA_F | TTC TGC CTT TTT GGG ATC AA | RDA (A) | |
| OTU171_RDA_R | CCT TGT TT A TTC AAG TGG GTG A | RDA (A) | |

(1). Alexa, P., K. Stouracova, J. Hamrik, and I. Rychlik. 2001. Veterinary Medicine 46(2): 46-49.
(2). Heilig, H., E. G. Zoetendal, E. E. Vaughan, P. Marteau, A. D. L. Akkermans, and W. M. de Vos. 2002. Applied and Environmental Microbiology 68(1): 114-123.
(3). Leser, T. D., J. Z. Amenuvor, T. K. Jensen, R. H. Lindecrona, M. Boye, and K. Moller. 2002. Applied and Environmental Microbiology 68(2): 673-690.
(4). Nübel, U., B. Engelen, A. Felske, J. Snaidr, A. Wieshuber, R. I. Amann, W. Ludwig, and H. Backhaus. 1996. Journal of Bacteriology 178: 5636-5643.

TABLE 3
DNA relatedness among L. sobrius sp. nov. and phylogenetically
closely related Lactobacillus species.

| | Reassociation (%) with DNA from strain: | | | |
|---|---|---|---|---|
| Strain | OTU171_001^{T} | OTU171_002 | OTU171_003 | JCM1039 |

L. sobrius

| | | | | |
|---|---|---|---|---|
| OTU171_001^{T} | 100 | 98 | 90 | 45 |
| OTU171_002 | 99 | 100 | 84 | 46 |
| OTU171_003 | 95 | 90 | 100 | 35 |
| OTU171_004 | 96 | 92 | 93 | 36 |
| OTU171_005 | 99 | 92 | 100 | 25 |
| OTU171_006 | 78 | 81 | 93 | 29 |
| L. kitasatonis JCM1039^{T} | 41 | 40 | 38 | 100 |
| L. amylovorus DSMZ 20531^{T} | 49 | 44 | 47 | 25 |
| L. crispatus DSMZ 20584^{T} | 2 | 3 | 2 | 18 |
| L. gallinarum DSMZ 10532^{T} | 22 | 24 | 31 | — |
| L. acidophilus DSMZ20079 | 13 | 12 | 13 | — |
| L. helveticus DSMZ 20075^{T} | 11 | 13 | 14 | — |

All values are mean values of two analyses with a standard deviation below 5%.
-
—, not tested.

TABLE 4
Effect of dietary supplementation with L. sobrius strain 001^{T} on growth,
feed intake, diarrhoea score (1 = feces very consistent; 5 = watery
diarrhoea), total E. coli and ETEC shedding of challenged pigs (least
squares means ± SEM).

| | | Control | LAB | SEM |
|---|---|---|---|---|
| Daily live weight gain | g | 101.3 | 176.2 | 21.8 * |
| Daily feed intake | g | 241.7 | 257.1 | 11.9 |
| Average diarrhoea score | | 2.46 | 2.84 | 0.18 |
| Days with | N | 2.83 | 4.74 | 0.61 * |
| diarrhoea score > 2 | | | | |

Feacal shedding:
Total E. coli

| | | | | |
|---|---|---|---|---|
| 2^{nd} day | Log10 (CFU g⁻¹) | 7.08 | 7.46 | 0.31 |
| 3^{rd} day | Log10 (CFU g⁻¹) | 7.87 | 8.05 | 0.29 |

ETEC (feces)

| | | | | |
|---|---|---|---|---|
| 2^{nd} day | Log10 (CFU g⁻¹) | 4.56 | 4.68 | 0.47 |
| 3^{rd} day | Log10 (CPU g⁻¹) | 4.97 | 6.17 | 0.55 |

* Effect of diet: p < 0.05.

TABLE 5
Effect of dietary supplementation with L. sobrius_001^{T}
strain on pH of different gastrointestinal tract segments
of ETEC challenged pigs (least squares means ± SEM)

| | Control | LAB | SEM |
|---|---|---|---|
| Stomach | 3.27 | 3.64 | 0.190 |
| Duodenum | 5.70 | 5.41 | 0.205 |
| Jejunum | 6.91 | 7.10 | 0.151 |
| Cecum | 5.88 | 5.74 | 0.088 |
| Colon | 6.15 | 6.11 | 0.065 |

TABLE 6
Comparison of quantitative real-time PCR, and FISH for detection and
quantification of L. sobrius species in four porcine ilea samples
(A, B, C, D). Counts are expressed as mean cells. g⁻¹.

| Sample | Real-time PCR | FISH |
|---|---|---|
| A | 2.14 × 10⁸ | 2.05 × 10⁸ |
| B | 1.69 × 10⁸ | 1.63 × 10⁸ |
| C | 1.53 × 10⁸ | 1.55 × 10⁸ |
| D | 1.09 × 10⁵ | — |

—, not detected, lower than 10⁵ cells. g⁻¹.

TABLE 7
Numbers of L. sobrius strain 001^{T}, L. sobrius species, ETEC, and total
bacteria (BAC) in ileal samples, as determined by real-time PCR.
Counts are expressed as mean ± SD cells g⁻¹.

| | Control diet | LAB diet |
|---|---|---|
| BAC | 9.3 ± 1.1 × 10⁹ | 5.4 ± 0.9 × 10⁹ |
| L. sobrius species | 2.3 ± 3 × 10⁴ | 1.6 ± 0.9 × 10⁸* |
| L. sobrius strain 001^{T} | <10³ | 0.66 ± 1 × 10⁸ |
| ETEC | 1 ± 1 × 10⁶ | 4.2 ± 0.7 × 10⁴* |

*significant differences from the respective values compared at p < 0.05.

### Sequence:

SEQ ID NO: 1—sequence used for targeting the 16S rRNA of L. amylovorus-like phylotype OTU171
SEQ ID NO: 2 to 5—primer for Lactobacillus specific PCR amplification
SEQ ID NO: 6 and 7—primer for total bacterial quantification
SEQ ID NO: 8 and 9—primer for enterotoxogenic E. coli quantification
SEQ ID NO: 10 and 11—primer for species specific quantitative PCR
SEQ ID NO: 12 and 13—primer for strain specific quantitative PCR

1. An isolated Gram-positive bacterium which comprises:
a) 16S rRNA which hybridises under stringent conditions to a nucleotide sequence according to SEQ ID NO. 1, and
b) genomic DNA which shows at least 75% DNA-DNA re-association with the genomic DNA of the Gram-positive bacterium deposited under accession number CBS 117345, whereby DNA-DNA re-association is determined by filter hybridization.

2. The isolated Gram positive bacterium according to claim 1, capable of fermenting D-raffinose, sucrose and lactose.

3. The isolated Gram positive bacterium according to claim 1 which is deposited under accession number CBS 117345 or a bacterium derived therefrom by cell division.

4. (canceled)

5. A composition comprising:
(a) a strain of an isolated Gram-positive bacterium comprising:
(i) 16S rRNA which hybridises under stringent conditions to a nucleotide sequence according to SEQ ID NO. 1, and
(ii) genomic DNA which shows at least 75% DNA-DNA re-association with the genomic DNA of the Gram-positive bacterium deposited under accession number CBS 117345, whereby DNA-DNA re-association is determined by filter hybridization; and
(b) a carbohydrate.

6-8. (canceled)

9. A method for weaning an animal comprising administering to the animal a composition comprising an isolated Gram-positive bacterium further comprising:
(a) 16S rRNA which hybridises under stringent conditions to a nucleotide sequence according to SEQ ID NO. 1, and
(b) genomic DNA which shows at least 75% DNA-DNA re-association with the genomic DNA of the Gram-positive bacterium deposited under accession number CBS 117345, whereby DNA-DNA re-association is determined by filter hybridization.

10-12. (canceled)

13. The composition according to claim 5 comprising at least 1×10⁷ colony forming units (cfu) of said strain.

14. The composition according to claim 5 further comprising probiotic bacteria, prebiotics, minerals, nutrients, flavourings, fat, protein, oligosaccharide carbohydrates, sugar beet pulp, or combinations thereof.

15. The composition according to claim 14 in which the oligosaccharide carbohydrate is fructoooligosaccharide.

16. A method of inhibiting or reducing pathogen infection and/or preventing or reducing diarrhoea in an animal, the method comprising administering to the animal a composition comprising an isolated Gram-positive bacterium further comprising:
(a) 16S rRNA which hybridises under stringent conditions to a nucleotide sequence according to SEQ ID NO. 1, and
(b) genomic DNA which shows at least 75% DNA-DNA re-association with the genomic DNA of the Gram-positive bacterium deposited under accession number CBS 117345, whereby DNA-DNA re-association is determined by filter hybridization.

17. The method according to claim 16 in which the animal is a monogastric animal.

18. The method according to claim 17 in which the animal is a cat, hamster, dog, or human being.

19. The method according to claim 16 in which the pathogen is Clostridium, Salmonella, Shigella, Yersinia, Escherichia, Enterobacter, Klebsiella, or a combination thereof.

20. The method according to claims 16, in which at least 1×10⁷ colony forming units (cfu) of the isolated Gram-positive bacterium is administered to the animal per day.

21. The method according to claims 20, in which at least 1×10⁸ colony forming units (cfu) of the isolated Gram-positive bacterium is administered to the animal per day.

22. The method according to claims 21, in which at least 5×10¹⁰ colony forming units (cfu) of the isolated Gram-positive bacterium is administered to the animal per day.

23. A method of detecting Gram-positive bacterium capable of inhibiting or preventing the growth of intestinal pathogens in a sample, the method comprising:
(a) obtaining a probe which includes a marker and a nucleotide sequence according to SEQ ID NO:1;
(b) contacting the probe with the sample under hybridising conditions;
(c) removing unbound probe, and
(d) detecting the marker indicating the presence of the Gram-positive bacterium.

24. The method according to claim 23 in which the sample is mammalian excrement.
